(19) United States
(12) Patent Application Publication
(10) Pub. No.: US 2004/0156912 A1
(43) Pub. Date: Aug. 12, 2004

(54) Surface functional member (member provided with surface layer of adsorbed functional particles)

(75) Inventors: Koichi Kawamura, Shizuoka-ken (JP); Takeyoshi Kano, Shizuoka-ken (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 10/771,227

(22) Filed: Feb. 4, 2004

(30) Foreign Application Priority Data
Feb. 5, 2003 (JP) 2003-28321

(51) Int. Cl. A61K 9/14

(52) U.S. Cl. 424/489

(57) ABSTRACT

The surface functional member of the present invention has a layer of adsorbed particles which is formed by adsorbing functional particles that are bondable with ionic polar groups onto the substrate on which graft polymer chains having the ionic polar groups are present. The graft polymer chains are preferably formed from a polymerization initiator fixed on the substrate surface by atom transfer radical polymerization. The surface functional member is provided with a layer of functional particles excelling in durability that are firmly adsorbed in the form of a single- or multi-layer structure on its surface so that the functions of the adsorbed functional particles can be exhibited for a long period of time.

### CROSS-REFERENCE TO RELATED APPLICATION

This application claims priority under 35 USC 119 from Japanese patent Application Nos. 2003-28321, the disclosures of which are incorporated by reference herein.

### BACKGROUND OF THE INVENTION

1. Field of the Invention

The present invention relates to a surface functional member, and more specifically, to a versatile surface functional member which is provided with a functional surface layer that is composed of adsorbed particles having various functions, such as a roughened surface member, a conductive member, and a light shielding member.

2. Description of the Related Art

Conventionally various kinds of members having a surface layer with various functions, the surface layer being formed by making functional particles be adsorbed onto a desired base member have been provided. Examples of members having a surface layer of adsorbed particles include: an antireflection member having a rough surface formed by making resin or metallic fine particles be adsorbed onto the surface; a conductive member having a surface with conductive particles adsorbed thereon; an antifouling and antimicrobial member having a surface with antimicrobial metal (oxide) particles adsorbed thereon; a gas barrier film having a surface with a number of particles adsorbed thereon in the form of a multi-layer structure which is used to decrease air permeability; and a light shielding member having a surface with particles for blocking ultraviolet rays, infrared rays, or visible light so as to reduce the transmittance of light having these wavelengths. These and other members having a surface with particles adsorbed thereon are an important technologies to achieve higher functions such as larger surface area, higher resolution, and higher densities in the fields of catalysts, recording materials, sensors, electronic devices, optical devices and the like. Therefore, they have been being studied enthusiastically.

A typical example of these surface functional members is a roughened surface member, which will be described below. The roughened surface member having unevenness in accordance with the diameter of the particles is useful as a material for controlling the reflective index at an interface so as to prevent light reflection.

In recent years, image displays typified by liquid crystal displays (LCD), plasma displays (PDP), cathode ray tube displays (CRT), and electroluminescence (EL) lamps have come to be used in various fields including televisions, computers, and various kinds of mobile devices which have become widely used in recent years, and these displays are making remarkable progress. These displays are expected to improve their performance including image quality and power consumption, while improving the functions of various kinds of devices in which these displays are used. For the improvement of the image quality, in addition to improving in video pixel density and the realization of bright color tone, antireflection performance for preventing the display screen from dazzling by light such as illumination is an important element.

In particular, portable terminal displays which have come into wide use in recent years are obviously intended to be used outdoors, and in such a condition of use, there is a growing demand for higher antireflection performance to prevent external light such as sunlight or fluorescence from being reflected from a display screen.

Moreover, LCDs which are characterized by being light-weight, compact, and versatile are now in wide use. Mobile devices (portable terminals) with LCDs mounted thereon and utilizing a touch panel system in which a specific region on the display screen is touched with a plastic pen or directly with a finger for operation are in wise use. In this system, durability such as abrasion resistance and antifouling properties are becoming important elements of the display surface, in addition to image quality and antireflection performance.

Antireflection has been generally realized by roughening the incident surface of light so as to scatter or diffuse light. Surface roughening processes generally used include: a process of directly roughening the surface of the base member by sand blasting, embossing or other methods, and a process of forming a roughened surface layer by applying a filler-containing coating solution onto the base member surface and drying the solution to make the filler be adsorbed onto the surface.

Above all, the process of forming a filler-containing roughened surface layer on the base member surface is being widely used at present because it is easy to control the size of unevenness on the roughened surface and also it is easily manufactured. Regarding this method, Japanese Patent Application Laid-Open (JP-A) No. 6-18706 shows a roughened surface layer containing a UV-curable resin and resin beads as components for use in highly transparent plastic film with poor heat resistance.

It has been also proposed to replace resin beads by an inorganic dye which is excellent in abrasion resistance such as silica; however, there is a problem that inorganic dyes do not have sufficient dispersibility, making it hard to form a homogeneous roughened surface layer. To overcome this problem, JP-A No. 11-287902 proposes a roughened surface layer using two different kinds of pigments which are made from silica and resin filler excellent in dispersibility.

However, in all of methods shown in the patent documents show filler used for the formation of unevenness is coated onto the base member with a binder, and there is a problem that the binder may lessen the unevenness of the filler, making it hard to obtain the designed antireflection performance. Furthermore, if the binder is diluted or decreased in amount in an attempt to improve the effects of unevenness of the filler, it may cause the film strength to decrease so as to deteriorate the durability.

As another method for forming the antireflection layer, it is known to accumulate a material having a high reflection index and another material having a low reflection index alternately to form a multi-layer structure. The multi-layer structure can be formed by a vapor phase process in which a film is formed by depositing a material with a low reflection index represented by SiO₂ and another material with a high reflection index such as TiO₂ or ZrO₂ alternately, the hydrolysis of metal alkoxide, sol-gel using condensation polymerization, or other methods.

These methods for forming the antireflection layer having the multi-layer structure have the following drawbacks. In the vapor phase method such as deposition, a processing device is expensive and a large-sized layer is hard to manufacture. In the case of forming the antireflection layer by the sol-gel, the production cost is high because coating and sintering is repeated. As another drawback, the obtained antireflection layer shows a violet or greenish color, which makes dirt noticeable.

With an improvement in the resolution of displays, the roughened surface layer is being required to be more precise in the height and spacing of the unevenness. Although the higher image quality can be achieved by havinng a higher density of pixels, when the spacing of the unevenness is larger than the pitch of the pixels, glare due to interference tends to occur, making it impossible to obtain the desired antireflection properties. Hence, the unevenness of the roughened surface layer are controlled in such a manner as to have no variations in the height and spacing, thereby providing an antireflection layer which is homogeneous and has high antireflection performance, regardless of the area of the image display.

As described above using the antireflection member as an example, it has been difficult to form a functional surface layer excellent in durability by making particles having a specific function be adsorbed onto a desired base member surface. For example, N. J. Nattan, M. Brust et al. have proposed a method for making gold particles be adsorbed in the form of multi layers onto the base member surface by repeating several times the process of adsorbing negatively charged colloidal gold particles onto the base member surface of silicon oxide, and forming a cross-linking structure by using of amino propane thiol as a linker so as to fix the particles on the surface. This technique, however, requires complex processes and therefore is unsuitable for the formation of a practical layer of adsorbed particles.

In some coating methods, the functional particles used for the formation of the functional surface layer lose their functions when the binder used to fix particles covers the surface or is present between particles so as to lessen the unevenness, thereby failing to fully exhibit the designed functions.

In view of these problems, it has been desired to provide a surface functional member with a layer of functional particles firmly adsorbed on its surface which are excellent in durability, have a single- or multi-layer structure, and also have long-lasting effects, or a surface functional member having functional particles adsorbed on its surface with a uniform thickness in a single- or multi-layer condition, the functional particles being excellent in durability and having long-lasting effects.

### SUMMARY OF THE INVENTION

As a result of studying properties of a base member having a graft polymer on a surface thereof, the present inventors discovered that, by introducing ionic polar groups into the graft polymer, there are strong absorption properties with respect to particles having a property of being able to interact with these ionic polar groups and it is possible to form and arrange particles that have specific properties at high density. By using this, the inventors discovered that a particle absorption layer, which utilizes the excellent properties of the particles, and completed the present invention.

Further, the inventors discovered that, by using atom transfer radical polymer method as a surface graft method, a graft layer of even thickness can be formed and, by using and adsorbing particles to this graft layer, a surface functional material, which has even thickness and at which particles are accumulated in a single or multiple layers, can be made, and completed the present invention.

The surface functional member of the invention is characterized in that a layer of adsorbed particles, which are bondable with ionic polar groups, is provided on a substrate having a surface at which graft polymer chains having ionic polar groups are present.

The graft polymer chains having the ionic polar groups which adsorb particles are preferably introduced by atom transfer radical polymerization with a polymerization initiator fixed on the substrate surface as a base.

The mechanism of the invention is not evedent, but it is estimatede as follows.

It is known that a polymer synthesized by atom transfer radical polymerization has extremely small distribution of molecular weight and a low degree of distribution. In the same manner, the invention also generates a graft polymer having small distribution of molecular weight and uniform molecular weight, thereby forming a graft layer having a uniform polymer film thickness. Hence, it is presumed that a functional particle layer having a homogeneous film quality can be obtained by making the graft polymer adsorb the particle.

### DETAILED DESCRIPTION OF THE INVENTION

The following is a detailed description of the surface functional member of the present invention.

The surface functional member of the invention has a substrate corresponding to the support member and at least one side of the substrate has a surface with graft polymer chains having ionic polar groups, and the surface with the ionic polar groups must be formed by atom transfer radical polymerization.

When the surface functional member of the invention is used as a light transmission member such as antireflection film or infrared rays adsorbing film, the supporting substrate is preferably a transparent substrate.

The surface functional member of the invention is preferably produced through each of the following processes.

1. A step of of image-wise fixing a polymerization initiator on the surface of a substrate;

2. A step of forming a graft polymer from the polymerization initiator by atom transfer radical polymerization with a monomer having ionic polar groups to form a pattern comprising regions having a graft polymer formed and not formed; and

3. A step of allowing the graft polymer to adsorb fine particles.

Well-known means shown in literature can be used to produce the surface functional member of the invention. The processes of producing the surface functional member of the invention will be described as follows, although these are not the only processes usable to produce the surface functional member of the invention.

1. A Step of of Image-Wise Fixing a Polymerization Initiator on the Surface of a Substrate

Any of the methods shown in the literature can be used as the process of fixing the initiator onto the substrate surface. From the viewpoint of operational facilitation and applicability to a large area, it is preferable to make the initiator having terminal groups bondable with a substrate such as a silane coupling agent be adsorbed onto the substrate surface, preferably onto the entire surface of the desired region.

(Subsrate)

The substrate applicable to the invention may be selected according to the use of the surface functional member. To be more specific, the substrate can be a plate made from inorganic material such as glass, silicon, aluminum, or stainless steel, or organic material such as a polymer compound.

The substrate made from inorganic material can also be a plate made from a metal such as gold, silver, zinc, or copper, or can have a surface with metal oxide thereon such as ITO, tin oxide, alumina, or titanium oxide.

Examples of the substrate made of an inorganic material include, substrates made of resin materials selected from polyethylene, polypropylene, polystyrene, cellulose diacetate, cellulose triacetate, cellulose propionate, cellulose butyrate, cellulose acetate butyrate, cellulose nitrate, polyethylene terephthalate, polycarbonate, polyvinyl acetal, polyurethane, epoxy resin, polyester resin, acrylic resin and polyimide resin. When the polymer substrate is used, a functional group such as hydroxyl group or carboxyl group may be introduced onto the surface of the substrate by corona treatment or plasma treatment in order to improve the binding of the substrate to an initiator having a reactive functional group.

(Polymerizatin Initiator)

The initiator may be any known compound having both a moiety that initiates polymerization upon exposure to light (also referred to hereinafter as “initiating site”) and a moiety that can be bonded to a substrate (also referred to hereinafter as “bonding site”) in the same molecule. For example, the following compounds can be mentioned.

Specifically, examples of the compound which can be introduced as the initiating site include compounds represented by the following general formulae As the initiator to be fixed on the substrate, any of the well-known materials can be used as long as it is a compound having in the same molecule a part to initiate polymerization by exposure (hereinafter referred to as initiating site) and a part to be bonded with the substrate (hereinafter referred to as bonding site). Such a polymerization initiator compound can be formed by introducing the partial structure containing the initiating site into a compound having the bonding site, or by other methods. As the initiator compound, the followings can be used.

As the initiating site, generally, an organic halide (for example, an ester compound having a halogen at the α-position or a compound having a halogen at a benzyl position) or a halogenated sulfonyl compound is introduced as a partial structure. A compound having a group working in place of halogen, for example a diazonium group, azido group, azo group, sulfonium group or oxonium group may also be used insofar as the compound has function as an initiator similar to the above halogenated compound.

Specifically, examples of the compound which can be introduced as the initiating site include compounds represented by the following general formulae

C₆H₅—CH₂X, C₆H₅—C(H)(X)CH₃,

C₆H₅—C(X)(CH₃)₂,

(wherein C₆H₅ represents phenyl group and X represents a chlorine atom, a bromine atom or an iodine atom.)

R¹—C(H)(X)—CO₂R²,

R¹—C(CH₃)(X)—CO₂R²,

R¹—C(H)(X)—C(O)R²,

R¹—C(CH₃)(X)—C(O)R².

(In the general formula R¹ and R² each independently represent a hydrogen atom, an alkyl group having 1-20 carbon atoms, an aryl group having 6-20 carbon atoms, or an aralkyl group having 7-20 carbon atoms, and X represents a chlorine atom, a bromine atom or an iodine atom.)

R¹—C₆H₄—SO₂X

(In the general formula R¹, has the same definition as the above definition of R¹, and X has the same definition as the above definition of X.)

From the viewpoint of stability with respect to time passage, the α-halogen ester compound is particularly preferable as the initiating site of the initiator. In the above examples, ester compounds each having a halogen atom at α position and compounds each having a halogen atom at a benzyl position are hydrophobic, while compounds each including solfonyl halide as a partial structure are hydrophilic.

The binding site in the initiator, that is, the substrate-binding group (functional group that can be bonded to a substrate) may be a thiol group, a disulfide group, an alkenyl group, a crosslinking silyl group, a hydroxyl group, an epoxy group, an amino group and an amide group. Particularly preferable substrate-binding group among these groups are a thiol group and a crosslinking silyl group.

Examples of the initiator having an initiating site and a binding site include, for example, compounds represented by the following general formula (1):

R⁴R⁵C(X)—R⁶—R⁷—C(H)(R³)CH₂—[Si(R⁹)_{2-b}(Y)_{b}]ₘSi(R¹⁰)₃₋ₐ(Y)ₐ (1)

[In the general formula (1), R³, R⁴, R⁵, R⁶ and R⁷ have the same definition as that of R¹ and R², and X has the same definition as the above definition of X. R⁹ and R¹⁰ each independently represent an alkyl group having 1-20 carbon atoms, an aryl group having 1-20 carbon atoms, an aralkyl group having 1-20 carbon atoms or a triorganosiloxy group represented by (R′)₃SiO— wherein R′ represents a monovalent hydrocarbon group having 1-20 carbon atoms, and the three R′ groups may be the same as or different from each other. When two or more R⁹ groups are present or two or more R¹⁰ groups are present, the groups may be the same as or different from each other.

Y represents a hydroxyl group, a halogen atom or a hydrolyzable group, and when two or more Y groups are present, the groups may be the same as or different from each other.

And a represents an integer of 0, 1, 2 or 3, b represents an integer of 0, 1 or 2, and m represents an integer of 0 to 19. Further, the relationship

a+mb≧1 is satisfied.

Among the compounds represented by the general formula (1), compounds represented by the following general formulae are preferable:

XCH₂C(O)O(CH₂)ₙSi(OCH₃)₃,

CH₃C(H)(X)C(O)O(CH₂)ₙSi(OCH₃)₃,

(CH₃)₂C(X)C(O)O(CH₂)ₙSi(OCH₃)₃,

(CH₃)₂C(X)C(O)O(CH₂)ₙSiCl₃,

XCH₂C(O)O(CH₂)ₙSiCl₃,

CH₃C(H)(X)C(O)O(CH₂)ₙSi(CH₃)(OCH₃)₂,

(CH₃)₂C(X)C(O)O(CH₂)ₙSiCl₃,

In the general formulae (8-1) to (8-7), X represents a chlorine atom, a bromine atom or an iodine atom, and n represents an integer of 0 to 20.

Other examples of the initiator having an initiating site and a binding site include compounds represented by the following general formula (2):

(R¹⁰)₃₋ₐ(Y)ₐSi—[OSi(R⁹)_{2-b}(Y)_{b}]ₘ—CH₂—C(H)(R³)—R¹¹—C—(R⁴)(X)R⁸—R⁵ (2)

In the general formula (2), R³, R⁴, R⁵, R⁷, R⁹, R¹⁰, a, b, m, X and Y respectively have the same definitions as defined above. R⁸ have the same definition as that of R¹ and R².

Among the compounds represented by the general formula (2), compounds represented by following general formulae are preferable:

(CH₃O)₃SiCH₂CH₂C(H)(X)C₆H₅,

Cl₃SiCH₂CH₂C(H)(X)C₆H₅,

Cl₃Si(CH₂)₂C(H)(X)—CO₂R,

(CH₃O)₂(CH₃)Si(CH₂)₂C(H)(X)—CO₂R,

(CH₃O)₃Si(CH₂)₃C(H)(X)—CO₂R,

(CH₃O)₂(CH₃)Si(CH₂)₃C(H)(X)—CO₂R,

In the general formulae, X represents chlorine, bromine or iodine, and R represents an alkyl group having 1-20 carbon atoms, an aryl group having 1-20 carbon atoms or an aralkyl group having 1-20 carbon atoms.

The initiator compound having the initiating site and the bonding site in the same molecule can be fixed on the substrate via the bonding site by merely being coated on the substrate.

2. A Step of Forming a Graft Polymer from the Polymerization Initiator with a Monomer Having Ionic Polar Groups on the Substrate Surface

In this step, the initiator in the step (1) of fixing an initiator to the surface of a substrate, graft polymerization is initiated and carried out by atom transfer radical polymerization with a monomer having ionic polar groups, thereby generating a graft having ionic polar groups and forming a graft polymerized layer.

Monomers used in the graft polymerization in the invention are preferably ionic polar monomers, which include the following hydrophilic monomers.

Some of general hydrophilic polymers usable in the invention can be obtained by polymerizing the following hydrophilic monomers: (meta)acrylic acid or its alkali metal salt and amine salt; itaconic acid or its alkali metal salt and amine salt; amide-based monomers such as 2-hydroxyethyl(meta)acrylate, (meta)acrylamide, N-monomethylol(meta)acrylamide, and N-dimethylol(meta)acrylamide; allylamine or its halide acid salt; 3-vinyl propionic acid or its alkali metal salt and amine salt; vinyl sulfonic acid or its alkali metal salt and amine salt; ethylene glycol-based monomers such as diethylene glycol(meta)acrylate, and polyoxy ethylene glycol mono(meta)acrylate; 2-sulfoethyl(meta)acrylate, 2-acrylamide-2-methyl propane sulfonic acid, acid phosphoxy polyoxy ethylene glycol mono(meta)acrylate, and salts there of.

The monomers used in graft polymerization in the invention include, in addition to the aforementioned ionic polar monomers, ionic monomers capable of forming ionic groups which are polar groups. These ionic monomers include positively charged monomers such as ammonium and phosphonium as mentioned above, and monomers which have an acid group such as sulfonic group, carboxyl group, phosphoric acid group, or phosphonic acid group, and which are either negatively charged or can be dissociated by negative charge.

The ionic monomers particularly useful in the invention include the following specific examples: vinyl sulfonic acid or its alkali metal salt and amine salt; vinyl styrene sulfonic acid or its alkali metal salt and amine salt; 2-sulfoethylene(meta)acrylate; 3-sulfopropylene(meta)acrylate or its alkali metal salt and amine salt; 2-acrylamide-2-methyl propane sulfonic acid or its alkali metal salt and amine salt; phosphoric acid monomers such as mono(2-acryloyloxy ethyl) acid phosphate, mono(2-methacryloyloxy ethyl) acid phosphate, acid phosphoxy polyethylene glycol mono(meta)acrylate; or their alkali metals and amine salts.

It goes without saying that the monomers usable in the invention are not limited to these examples.

(Method for Graft Polymerization)

The invention is characterized by applying atom transfer radical polymerization to formation of the graft polymer. Hereinafter, atom transfer radical polymerization is briefly described.

(Outline of Atom Transfer Radical Polymerization)

In usual radical polymerization, since the rate of polymerization is high and the reaction is easily terminated by coupling of radicals, it is considered difficult to regulate the molecular weight of the polymer. However, it is known that when “living radical polymerization method” is employed, the reaction is hardly terminated. Accordingly, polymers having narrow molecular-weight distribution (Mw/Mn of about 1.1 to 1.5) can be obtained, and the control of the molecular weight can be easily achieved by the monomer/initiator ratio.

Among the “living radical polymerization methods”, “atom transfer radical polymerization method”, in which a vinyl monomer is polymerized in the presence of an organic halide or a halogenated sulfonyl compound as an initiator and a transition metal complex as a catalyst, is preferable for producing a vinyl polymer having a specific functional group. This is because “atom transfer radical polymerization method” has higher degree of freedom of design of the initiator and catalyst in addition to the characteristics of “living radical polymerization methods” since the initiator has a halogen group or the like at its terminal which group is fairy advantageous to a functional group exchange reaction.

As the atom transfer radial polymerization method, mention is made of methods described by Matyjaszewski et al. in Journal of American Chemical Society (J. Am. Chem. Soc.) 1995, vol. 117, page. 5614; Macromolecules, 1995, vol. 28, page 7901; Science, 1996, vol. 272, page 866; WO96/30421; WO97/18247; WO98/01480; WO98/40415; by Sawamoto et al, in Macromolecules, 1995, vol. 28, page 1721; JP-A Nos. 9-208616 and 8-41117.

The term “atom transfer radical polymerization” used herein refers not only to usual atom transfer radical polymerization using an organic halide or a halogenated sulfonyl compound as an initiator as described above, but also to “reverse atom transfer radical polymerization”, in which a general initiator for free radical polymerization such as peroxide is combined with a usual atom transfer radical polymerization catalyst such as a copper (II) complex in a highly oxidized state.

(Atom Transfer Radical Polymer Catalyst)

The transition metal complex used as a catalyst in atom transfer radical polymerization is not particularly limited, and the catalysts described in International Publication No. WO 97/18247 can be utilized. Examples of Particularly preferable metal complex include complexes of 0-valent copper, monovalent copper, divalent copper, divalent ruthenium, divalent iron and divalent nickel.

In particular, copper complexes are preferable. Examples of monovalent copper compounds include cuprous chloride, cuprous bromide, cuprous iodide, cuprous cyanide, cuprous oxide, and cuprous chlorate. A tristriphenyl phosphine complex of divalent ruthenium chloride (RuCl₂(PPh₃)₃) is also a preferable catalyst. When a ruthenium compound is used as the catalyst, a kind of aluminum alkoxide is added as the activator. Other preferable catalysts are a bistriphenyl phosphine complex of divalent iron (FeCl₂(PPh₃)₂), a bistriphenyl phosphine complex of divalent nickel (NiCl₂(PPh₃)₂), and a bistributyl phosphine complex of divalent nickel (NiBr₂(PBU₃)₂).

When a copper compound is used as the catalyst, the ligands shown in PCT/US96/17780 can be used. Although not a restriction, amine-based ligands are usable. And preferable amine-based ligands are: 2,2′-bipryidyl and its derivative; 1,10-phenanthroline and its derivative; and aliphatic amines such as trialkyl amine, tetra methyl ethylene diamine, pentamethyl diethylene triamine, hexamethyl(2-aminoethyl) and others. In the invention, of these examples, aliphatic polyamines such as penta methyl diethylene triamine and hexamethyl(2-aminoethyl) amine are preferable.

The amount of ligand to be used is determined by the number of coordinations of transition metal and the number of groups where the ligands are positioned under the ordinary atom transfer radical polymerization. And these are set to be nearly equal. For example, 2,2′-biprlyidyl and its derivative is added to CuBr in a mole ratio of 1:2, and penta methyl diethylene triamine is added in a mole ratio of 1:1.

In the invention, in the case where ligands are added to initiate polymerization and/or to control catalyst activity, it is preferable that metal atoms exceed the ligands in number although it is not a restriction. The ratio of the coordinations to the groups to be coordinated is preferably not less than 1.2, more preferably not less than 1.4, particularly preferably not less than 1.6, and most preferably not less than 2.

(Reaction Solvent)

In the invention, graft polymerization reaction can be carried out in the absence or presence of solvents.

Examples of Solvents usable for the polymerization reaction include hydrocarbon solvents such as benzene and toluene; ether solvents such as diethyl ether, tetrahydrofuran, diphenyl ether, anisole, dimethoxy benzene; halogenated hydrocarbon such as methylene chloride, chloroform, and chlorobenzene; ketone solvent such as acetone, methyl ethyl ketone, and methyl isobutyl ketone; alcohol solvents such as methanol, ethanol, propanol, isopropanol, n-butyl alcohol, and tert-butyl alcohol; nitrile solvents such as acetonitrile, propionitrile, and benzonitrile; ester solvents such as ethyl acetate and butyl acetate; carbonate-based solvents such as ethylene carbonate and propylene carbonate; and water. These solvents can be used alone or in combination of two or more thereof.

In general, the graft polymerization reaction using a solvent is carried out by adding a monomer and a catalyst if necessary into the solvent and then soaking the substrate with the initiator fixed thereon in the solvent to be reacted for a prescribed period of time.

The graft polymerization reaction without solvent is generally carried out either at room temperature or under a condition of being heated up to 100° C.

When the surface functional member thus obtained is used as a roughened surface member for antireflection material, in an image display equipped with high density pixels for high resolution or a small-sized mobile image display with high resolution, it is preferable to use a transparent base member having surface smoothness so as to control the unevenness of the surface to be formed. However, in order to improve the macro antireflection performance, it is possible to previously roughen the base member surface in an attempt to increase the surface area, thereby introducing a larger number of ionic groups.

To roughen the base member surface, a well-known method suitable to the properties of the base member can be selected. To be more specific, when the base member is resin film, it is possible to use glow discharge process, spattering, sand blasting, buffing, particle adhering, particle coating, or the like. When the base member is a metal plate such as an aluminum plate, the surface can be roughened mechanically, melted and roughened electrochemically, or selectively melted chemically. As a mechanical method, it is possible to use a well-known method such as balling, brushing, blasting, or buffing. As another method, the electrochemical surface roughening method can be carried out in hydrochloric acid or electrolyte of nitrate by using AC or DC current. It is also possible to use both in combination.

3. Process of Adsorbing Particles onto Graft Polymerized Layer thus Obtained

According to the invention, the functional surface is obtained by making functional particles be adsorbed onto the ionic polar groups in the graft polymerized layer formed in the previous process. The functional particles used here will be described as follows.

[Particles Having the Properties Which Enable the Particles to Have Interaction with Ionic Polar Groups and to be Bonded Therewith]

(1) Examples of Particles

Next, the particles having the properties which enable the particles to be bonded with the ionic polar groups will be described as follows. The particles to be used can be selected depending on the purpose of use of the functional surface. The diameter of the particles also can be selected depending on the purpose. In the preferred embodiments of the invention, particles are adsorbed ionically, so it goes without saying that the diameter of the particles and the amount to be adsorbed are restricted according to the surface charge of the particles and the number of ionic groups. In general, the diameter is preferably in the range of 0.1 nm to 1 μm, and more preferably in the range of 1 to 300 nm, and particularly preferably in the range of 5 to 100 nm.

In the invention, if ionic polar groups are taken up as an example, the particles to be bonded by the interaction with the ionic polar groups of the graft polymer in the interface of the graft polymerized layer may be regularly arranged in a single layer condition or each particle of nano scale may be adsorbed to the respective ionic polar group of long graft chains, thereby being arranged in a multi-layer condition, according to the condition of the ionic polar groups in the graft layer.

The functional particles usable for the present invention will be described as follows in accordance with the purposes of the surface functional member.

(1-1) Particles for Antireflection Member

When the functional member of the present invention is used as an antireflection member, it is preferable that at least one kind of the particles selected from resin particles and metal oxide particles is used as the functional particles. The use of such particles can provide a roughened surface member which has homogeneous and excellent antireflection performance preferably used for an image display surface; which can obtain bright images without decreasing the image contrast; and which is suitable to the antireflection material having excellent durability.

The resin particles used for the antireflection member use an organic polymer at then center which is called a core, and the metallic oxide particles used for the antireflection member are preferably a metallic oxide selected from silica (SiO₂), titanium oxide (TiO₂), zinc oxide (ZnO), tin oxide (SnO₂), etc. It is also possible to use as pigment particles so-called a transparent pigment or white pigment such as calcium carbonate, aluminum hydroxide, magnesium hydroxide, clay, or talc, as long as they have the preferable pattern described below.

The resin particles have preferably a high degree of hardness from the viewpoint of durability, and specifically are spherical particles made from resin such as acrylic resin, polystyrene resin, polyethylene resin, epoxy resin, or silicon resin. Above all, cross-linking resin particles are particularly preferable.

In this purpose of use, the diameter of the particles is preferably in the range of 100 to 300 nm, and more preferably in the range of 100 to 200 nm. In the invention, the particles to be ionically bonded with the graft interface are arranged regularly in an almost single-layer condition. When the roughened surface member of the present invention is used as antireflection material, it is preferable from the viewpoint of effects to set the film thickness to λ/4 with respect to the wavelength (λ) whose reflection should be prevented. Considering that the diameter of the particles becomes nearly the same as the thickness of the roughened surface layer, when the diameter is smaller than 100 nm, the roughened surface layer becomes too thin and decreases the antireflection properties, whereas when the diameter is larger than 300 nm, the diffuse reflection gets larger and causes a more whitish state. This makes it hard to obtain a sense of transparency, and reduces the contact area where the particles are ionically bonded with the graft interface, so that the strength of the roughened surface layer tends to decrease.

(1-2) Particles for Conductive Film

When the functional member of the invention is used as conductive film, it is preferable to use at least one kind of particles selected from conductive resin particles, conductive or semiconductive metal particles, metal oxide particles, and metal compound particles.

As the conductive metal particles or the metal oxide particles, conductive metal compound powder having a specific resistance value of not more than 1×10³ Ω·cm can be used widely. To be more specific, it is possible to use silver (Ag), gold (Au), nickel (Ni), copper (Cu), aluminum (Al), tin (Sn), lead (Pb), zinc (Zn), iron (Fe), platinum (Pt), iridium (Ir), osmium (Os), palladium (Pd), rhodium (Rh), ruthenium (Ru), tungsten (W), molybdenum (Mo), alloys of these materials, tin oxide (SnO₂), indium oxide (In₂O₃), ITO (Indium Tin Oxide), ruthenium oxide (RuO₂), etc.

It is also possible to use metal oxide, metal compound particles having semiconducting properties. These are specifically as follows: oxide semiconductive particles such as In₂O₃, SnO₂, ZnO, Cdo, TiO₂, CdIn₂O₄, Cd₂SnO₂, Zn₂SnO₄, and In₂O₃—ZnO; particles doped with impurities suitable for these materials; spinel compound particles such as MgInO and CaGaO; conductive nitride particles such as TiN, ZrN, and HfN; and conductive boride particles such as LaB. These can be used either singly or as mixtures of two or more kinds.

(1-3) Particles for Surface Antimicrobial Material

When the functional member of the invention is used as antimicrobial material, it is preferable to use as the functional particles, metal or metal oxide particles having antimicrobial or sterilizing effects.

The materials which can form such metal (compound) particles specifically include: metals in simple substance having sterilizing properties such as silver (Ag) and copper (Cu); alloys containing at least one kind of these metals; and oxides of these metals. The materials also include metal oxide semiconductors, such as titanium oxide, iron oxide, tungsten oxide, zinc oxide, strontium titanate, and metal compounds mixed with platinum, gold, palladium, silver, copper, nickel, cobalt, rhodium, niobium, tin, etc, which exhibit sterilizing effects by the irradiation of light containing the wavelength of ultraviolet region such as fluorescent lamp or sunshine.

(1-4) Particles for Ultraviolet Adsorbing Member

When the functional member of the invention is used as an ultraviolet adsorbing member, it is preferable to use as the functional particles, metal oxide particles such as iron oxide, titanium oxide, zinc oxide, cobalt oxide, chromium oxide, tin oxide, or antimony oxide in order to have a high light shielding function in the regions of ultraviolet rays A and B regions (light wavelength: 280 to 400 nm). In the invention, a polymer compound is used as the base member and combined with the particles to exhibit high function and processability as the ultraviolet blocking film sheet, thereby being expected to have various applications. It is also expected to improve light stability of the polymer material by using the ultraviolet blocking effects of the metal oxide.

(1-5) Particles for Optical Material

The functional particles used in color filter, sharp cut filter, and nonlinear optical material for use in optical devices can be semiconductors such as CdS and CdSe or particles made from a metal such as gold (Au). As the base member, silica glass or alumina glass can be preferably used in a color filter or the like. It has been recently recognized that a combination of such a base member and a particle layer has a high third-order optical nonlinear susceptibility, so this functional material is expected to be used as nonlinear optical material for use in optical switches or optical memory.

The particles used in this case include: noble metals such as gold, platinum, silver, and palladium and alloys of these metals, and it is preferable from the viewpoint of safety to use particles made from material which is not quickly dissolved in alkali, such as gold or platinum.

The ultrafine particles of a metal or metal compound suitable as nonlinear optical material include ultrafine particles with an average diameter of 10 to 1000 angstrom such as gold (Au), silver (Sg), copper (Cu), platinum (Pt), palladium (Pd), rhodium (Rh), osmium (Os), iron (Fe), nickel (Ni), and ruthenium (Ru) in simple substance, and alloys containing as least one kind of these metals. The particle diameter can belong either to primary particle or to secondary particle; however, it is preferable not to cause scattering of the visible light. Particularly preferable particles are noble metal particles which are selected from Au, Pt, Pd, Rh, and Ag, and metal particles selected from Ti, V, Cr, Mn, Fe, Ni, Cu, Zn, Cd, Y, W, Sn, Ge, In, and Ga which can be independently dispersed in a solvent such as toluene and have a diameter of not more than 10 nm.

When nonlinear optical material is produced by using these ultrafine particles by an ordinary method such as sol-gel, impregnation, spattering, ion injection, or melting deposition, the easy agglomeration of the particles makes it hard to increase the concentration of the particles in the complex or decreases the productivity. In particular, particles having a low concentration and a small rate of contribution to the physical properties can be used in a restricted way and are not suitable for image memory or light integration circuit using third-order nonlinear optical effects. According to the structure of the invention, the particles are directly ionically bonded with the ionic groups on the base member surface, and the ionic groups are present in high density because of the grafts. Therefore, it is easy to increase the concentration of the particles, and the particles are particularly suitable for use in such nonlinear optical material in optical materials.

(1-6) Particles for Gas Barrier Film

When the surface functional member of the invention is used as gas barrier film, it is preferable to use as the functional particles, ultrafine particle powder made from an inorganic compound such as silicon oxide, zirconium oxide, titanium oxide, alumina, magnesium oxide, or tin oxide or made from a metal such as aluminum, tin, or zinc. The average diameter of such ultrafine particle powder is preferably not more than 100 nm, and more preferably not more than 50 nm. The ultrafine particle powder can be used in the form of one kind or a mixture of two or more kinds selected from the aforementioned inorganic compounds and metals. The use of an insulating inorganic compound such as silicon oxide as the ultrafine particle powder enables the whole functional member to have insulation. Silicon oxide is particularly preferable as the ultrafine particle powder because it is easily formed into ultrafine particle powder.

As the base member, it is preferable to use organic resin film with high gas barrier properties such as polyethylene terephthalate, polyamide, polypropylene, ethylene-vinyl alcohol copolymer, or polyvinyl alcohol.

(1-7) Particles for Organic Electroluminescent Element

Particles containing agglomerated organic dye molecules which emit light by the excitement of hot carriers can be used as the particles, and a layer of these particles can be formed on the base member surface having electrodes to form an organic electroluminescent element. The organic dyes used in this case are mentioned below; however, these are not the only dyes usable, and various kinds can be selected depending on the purpose of use of the solid state optical functional device.

The usable organic dyes include: oxazole-based dyes with blue light emission such asp-bis [2-(5-phenyloxazole)]benzene (POPOP); coumarin-based dyes with green light emission such as coumarin 2, coumarin 6, coumarin 7, coumarin 24, coumarin 30, coumarin 102, and coumarin 540; rhodamine-based (red) dyes with red color emission such as rhodamine 6G, rhodamine B, rhodamine 101, rhodamine 110, rhodamine 590, and rhodamine 640; oxazine-based dyes such as oxazine 1, oxazine 4, oxazine 9, and oxazine 118 which can provide emission in the near-infrared region and particularly suitable for optical functional device matching with optical communication.

In addition, cyanine-based dyes such as phthalocyanine and a cyanine iodide compound can be used. In selecting these dyes, it is preferable to select those easily dissolved in a polymer like acrylic resin for the purpose of forming a thin film. Such dyes include: POPOP, coumarin 2, coumarin 6, coumarin 30, rhodamine 6G, rhodamine B, and rhodamine 101.

The particles to be used can be organic molecules used for an organic electroluminescence (EL) film such as 8-hydroxy quinoline aluminum (AlQ₃), 1,4-bis-(2,2 diphenyl vinyl) biphenyl, a polyparaphenylene vinylene (PPV) derivative, a distyryl arylene derivative, a styryl biphenyl derivative, a phenanthroline derivative, or particles made by a solvent composed of the organic molecules and an additive.

The aforementioned (1-1) to (1-7) have described applied examples of the surface functional member of the invention and specific examples of the particles used preferably in the fields; however, the invention is not restricted to these examples. It goes without saying that graft polymer chains are generated by atom transfer radical polymerization by introducing polar groups such as ionic groups at least on one side of the base member, and the different kinds of particles which have the properties enabling the particles to be bonded with the ionic groups are selected and combined properly so as to compose various kinds of members having a functional surface having the properties of functional particles.

(2) About the Properties (Charges to be Ionically Bondable with Ionic Groups) of the Particle Surface

Of the various kinds of particles, the kinds having charges themselves such as silica particles can be adsorbed as they are onto a layer of adsorbed particles by selecting material for the surface having ionic polar groups, thereby introducing ionic polar groups opposite to the charges of the particles onto the support member surface. Regardless of the presence or absence of charges of the particles, particles having charges in high density are formed on the surface by a well-known method for the purpose of being bonded with the ionic polar groups present on the base member surface so as to be adsorbed to the introduced ionic polar groups. The latter method, that is, to provide the surface of the particles with arbitrary charges can have a wider variety of particles to be adsorbed.

These particles are preferably bonded at the maximum amount to be adsorbed to the ionic groups present on the support member surface from the viewpoint of durability. From the viewpoint of the efficiency of the appearance of functionality in the functional surface, the dispersion concentration of the dispersion solution is preferably about 10 to 20% by mass.

In the base member having ionic groups on its surface, the particles can be bonded with the ionic groups to form a layer of adsorbed particles by coating a dispersion solution of particles having charges on their surface onto the base member surface having graft polymer layers or ionic groups; soaking a film base member having ionic groups on its surface into the dispersion solution of particles having charges on their surface, or other methods. Whether the coating or the soaking is used, supplying an excess amount of charged particles can introduce the particles by the sufficient ionic bonding with the ionic groups. Therefore, the contact time between the particle dispersion solution and the base member having ionic groups on its surface is preferably about 10 seconds to 180 minutes, and more preferably about 1 to 100 minutes.

(3) A Step of Allowing the Graft Polymer to Adsorb Fine Particles

One specific example of the adsorption is as follows. While using a monomer having ionic groups such as positively charged ammonium as the polar group, graft polymer chains having ionic polar groups on the support member surface are introduced. Then, this base member is soaked in a dispersion solution of silica particles and then, an extra amount of dispersion solution is washed off with water. The result is a layer of adsorbed particles formed on the surface of the transparent base member in such a manner that silica particles are adsorbed closely in a single- or multi-layer condition according to the density of the ionic groups.

In this manner, the ionic polar groups are introduced on the base member and the particles are adsorbed thereon, thereby providing a layer of adsorbed particles having a desired function. Although the thickness of the layer of adsorbed particles can be selected according to the purpose, it is preferably in the range of 0.001 to 10 μm, more preferably in the range of 0.01 to 5 μm, and most preferably in the range of 0.1 to 2 μm. When the film is too thin, scratch resistance tends to decrease, and when it is too thick, transparency tends to decrease.

According to the surface functional member of the invention, a layer of particles having a specific function, such as metal oxide particles typified by silica are uniformly adsorbed to the ionic polar groups introduced onto the substrate electrostatically in high density. The layer of particles is formed without using a binder and also in a manner that graft polymer chains having ionic groups have a uniform molecular weight by atom transfer radical polymerization, and the surface layer of particles adsorbed in a single- or multi-layer condition. Therefore, the obtained functional surface has uniform thickness and properties, directly reflecting the properties of the particles.

When particles for the roughened surface member are used as the particles, a roughed surface layer is formed in such a manner that the particles are arranged to form uniform unevenness and the unevenness is uniform and fine. Furthermore, when this roughened surface member is used as the antireflection material, regardless of the achieved high antireflection performance, the layer itself is so thin that the use of a transparent substrate as the substrate (support member) can eliminate the fear of disturbing light transmittance. Consequently, it can be applied not only to the reflection type image displays but also the transmission type image displays.

The proper selection of the functional particles enables the formation of a layer of adsorbed particles capable of reflecting the properties of the functional particles onto a desired base member surface by applying a comparatively simple treatment. Furthermore, the layer of adsorbed particles exhibiting excellent functioning properties has excellent homogeneity and durability, so it can be applied to the aforementioned various purposes.

The particles have the following specific uses. The use of conductive organic or inorganic particles can provide the functional surface with electronic and electric functions; the use of magnetic particles such as ferrite particles can provide magnetic functions; the use of particles which adsorb, reflect, or scatter a specific wavelength of light can provide optical functions. Thus, different particles can provide different functions on the functional surface, thereby being utilized in a wide range of fields such as industrial products, medical products, catalysts, varistor (variable resistor), paints, and cosmetic products. In addition to the various kinds of functions of various kinds of particle composing materials, the use of polymer materials as the base member enables the use of the easy processability of the polymer materials, with an expectation of the development of novel materials.

The specific examples of the aforementioned wide range of use include: optical parts; sunglasses; light shielding film, light shielding glass, light shielding windows, light shielding containers, light shielding plastic bottles and other light shielding products against ultraviolet rays, visible light, and infrared rays; antimicrobial film; microbial disinfecting filter; antimicrobial plastic molding; fish nets; TV parts, phone parts, OA appliances parts, electric cleaner parts, electric fan parts, air conditioner parts, refrigerator parts, washing machine parts, humidifier parts, dish drier parts and other household electrical appliance parts; sanitary products such as toilet seat parts and washstand parts; building materials; vehicle parts; daily necessities; toys; and household goods.

### EXAMPLES

The present invention will be described specifically using the following embodiments; however, the present invention is not restricted to these embodiments.

### Example 1

[Formation of Supporting Substrate Having Ionic Polar Groups on its Surface]

(Fixing Initiator onto Silicon Substrate)

Silane coupling agent: (5-trichlorosilyl pentyl)-2-bromo-2-methyl propionate was synthesized by the method shown in the following reference: C. J. Hawker et al., Macromolecules 1999, 32 p.1424.

A silicon plate which was used as the substrate was soaked overnight in Piranha liquid (H₂SO₂:H₂O₂=3:1), washed sufficiently with deionized water and stored in water. Under an argen current, the silicon plate, which had been taken out of the water, was dried with nitrogen until moisture on the surface was removed, and then soaked overnight in a 1% dehydrated toluene solution of the silane coupling agent under an arogen current. Then, the silicon plate was taken out and washed with toluene and methanol. The result was a silicon substrate having a terminal silane coupling agent fixed on its surface as the initiator.

(Generation of Graft Polymer Chains by Atom Transfer Radical Polymerization of Acrylic Acid from Substrate with Fixed Initiator)

55.2 g of ion-exchanged water was put in a 1-liter separable flask, and 16 g (0.40 mol) of sodium hydroxide was added and dissolved therein. Then, drops of 28.8 g (0.40 mol) of acrylic acid were slowly dropped in this solution under an ice bath so as to regulate it at pH7. Under a current of Ar, 0.891 g (9.0 mmol) of copper chloride (I) and 3.12 g (20.0 mmol) of 2,2′-bipryidyl were added to this solution and stirred until they became homogeneous.

The silicon wafer produced by the aforementioned method was soaked in the solution and stirred overnight. After reacting stopped, the wafer was washed with water. The surface of the wafer was scrubbed and cleaned with cloth (BEMCOT manufactured by Asahi Chemical Industry Co., Ltd.) soaked with methanol so as to obtain a substrate “A” having graft polymer chains on the surface. The film thickness was measured with ellipsometry (VB-250, manufactured by J. A. Woollam) and the graft was found to have a film thickness of 100 nm. Several spots measured by ellipsometry had substantialy the same thickness, which revealed that a graft film with a uniform thickness had been formed.

(Absorption of TiO₂ Particles onto Substrate “A” Having Graft Polymer layers)

The substrate “A” which has surface having graft polymer layers was soaked in an aqueous dispersion of TiO₂ particles having positive charges (1.5% by mass, manufactured by C.I. KASEI Company Ltd.) for one hour, taken out, washed well with water, and scrubbed 30 times back and forth in the water by hand using a cloth (BEMCOT, manufactured by Asahi Kasei Corporation). Then, the base member was dried to form a member “B” having fine unevenness (roughened surface member “B”).

[Estimation of Abrasion Resistance]

The roughened surface member “B” thus obtained was scrubbed 30 times back and forth by hand using a cloth (BEMCOT, manufactured by Asahi Kasei Corporation) dampened with water. Before and after the scrubbing treatment, the surface was observed with a transmission type electron microscope (JEOLJEM-200CX) having a magnifying power of 100,000, and minute unevenness resulting from the particles were observed on the surface both before and after the scrubbing treatment. It was confismed that the minute unevenness on the surface was not damaged by the scrubbing.

The zeta potential of the TiO₂ particles was measured with zetasizer 2000 manufactured by Marvern Instruments and found to be +42 mV, which was a positive charge.

[Estimation of Antireflection Performance]

A ratio (φᵣ/φᵢ) of light flux φᵢ incident on the roughened surface member “B” to the light flux φᵣ reflected from the same surface, that is, a luminous reflectance (%) was measured with a photo spectroscope the roughened surface member “B” was found to have a luminous reflectance of 0.3%, namely excellent antireflection performance.

### Example 2

(Absorption of Al₂O₃ Particles onto Base Member “A” Having Graft Polymer Layer)

The same operation as Embodiment 1 was conducted except for the use of an aqueous dispersion of Al₂O₃ (manufactured by C.I. KASEI Company Ltd.) having a positive charge (1.5% by mass). The cross section of accumulated particles was observed with a scan-type electron microscope to find that Al₂O₃ had accumulated with a uniform thickness in the graft layer. After the scrubbing treatment was repeated in the same manner as in Embodiment 1, no change was observed, the layer of adsorbed particles, indicating that the layer of adsorbed particles was not damaged by the scrubbing. The aqueous dispersion of Al₂O₃ had a zeta potential of +77 mV.

### Comparative Example 1

(Absorption of ZnO Particles onto Base Member “A” Having Graft Polymer Layer)

The same operation as Example 1 was conducted except for the use of ZnO (manufactured by C.I. KASEI Company Ltd.) having a negative charge. The surface was observed with a scan-type electron microscope to find that ZnO hardly had been adsorbed in the graft film. The zeta potential of ZnO was −60 mV.

### Comparative Example 2

(Absorption of SiO₂ Particles onto Base Member “A” Having Graft Polymer Layer)

The same operation as Comparative Example 1 was conducted except for the use of SiO₂ (manufactured by C.I. KASEI Company Ltd.) having a negative charge. The surface was observed with a scan-type electron microscope to find that SiO₂ was hardly adsorbed in the graft film. The zeta potential of SiO₂ was −50 mV.

Comparative Examples 1 and 2 indicate that the particles having opposite charges to the graft polymers do not accumulate on the base member and that it is preferable to make the polarities of the graft polymers and the particles opposite from each other.

The invention provides a surface functional member which is provided with a layer of functional particles that are excellent in durability and firmly adsorbed on the surface of the member in a single- or multi-layer structure, the layer of adsorbed functional particles being able to be formed easily and the effects of the adsorbed functional particles having long-lasting effects.

What is claimed is:

1. A surface functional member comprising a layer of adsorbed particles, the layer being formed by adsorbing particles bondable with ionic polar groups onto a substrate surface containing graft polymer chains having ionic polar groups, which have been formed by atom transfer radical polymerization from a polymerization initiator fixed on the substrate surface.

2. A surface functional member of claim 1, wherein the graft polymer chains are formed, by atom transfer radical polymerization, using the initiator fixed on the substrate surface as a starting point by atom transfer radical polymerization.

3. A surface functional member of claim 1 obtained by
fixing a polymerization initiator onto the substrate surface;
forming a graft polymerization layer by generating a graft having ionic polar groups, wherein the initiator fixed on the substrate surface is used as a starting point, and graft polymerization is initiated and carried out by atom transfer radical polymerization using monomers having ionic polar groups; and
adsorbing the particles to the obtained graft polymerization layer.

4. A surface functional member of claim 1, wherein the polymerization initiator fixed onto the substrate surface is a compound which has an initiating site that initiates polymerization by exposure and a bonding site that is bondable with the substrate in a same molecule.

5. A surface functional member of claim 4, wherein the initiator contains an organic halide or a halogenated sulfonyl compound introduced as the initiating site in the molecule.

6. A surface functional member of claim 4, wherein the initiator contains an α-halogen ester compound introduced as the initiating site in the molecule.

7. A surface functional member of claim 4, wherein the initiator contains as the bonding site in the molecule at least one kind selected from the group consisting of thiol groups, disulfide groups, alkenyl groups, cross-linking silyl groups, hydroxyl groups, epoxy groups, amino groups, and amide groups.

8. A surface functional member of claim 1, wherein the initiator is a compound expressed by general formula (1) or general formula (2) below:
R⁴R⁵C(X)—R⁶—R⁷—C(H)(R³)CH₂—[Si(R⁹)_{2-b}(Y)_{b}O]ₘ—S(R¹⁰)₃₋ₐ(Y)ₐ (1)
wherein in general formula (1), R³, R⁴, R⁵, R⁶ and R⁷ each independently represents a hydrogen atom, an alkyl group having 1-20 carbon atoms, an aryl group having 6-20 carbon atoms, or an aralkyl group having 7-20 carbon atoms, and X represents a chlorine atom, a bromine atom or an iodine atom; R⁹ and R¹⁰ each independently represent an alkyl group having 1-20 carbon atoms, an aryl group having 1-20 carbon atoms, an aralkyl group having 1-20 carbon atoms or a triorganosiloxy group represented by (R′)₃SiO— wherein R′ represents a monovalent hydrocarbon group having 1-20 carbon atoms, and the three R′ groups may be the same as or different from each other; when two or more R⁹ groups are present or two or more R¹⁰ groups are present, the groups may be the same as or different from each other. Y represents a hydroxyl group, a halogen atom or a hydrolyzable group, and when two or more Y groups are present, the groups may be the same as or different from each other; a represents an integer of 0, 1, 2 or 3; b represents an integer of 0, 1 or 2; and m represents an integer of 0 to 19, wherein the relationship a+mb≧1 is satisfied;
(R¹⁰)₃₋ₐ(Y)ₐSi—[OSi(R⁹)_{2-b}(Y)_{b}]ₘ—CH₂—C(H)(R³)—R¹¹C—(R⁴)(X)R⁸—R⁵ (2)
wherein in general formula (2), R³, R⁴, R⁵, R⁷, R⁹, R¹⁰, a, b, m, X and Y respectively have the same definitions as defined general formula (1); and R⁸ has the same definition as that of R¹ and R².

9. A surface functional member of claim 3, wherein the monomer having ionic polar groups used for the formation of the graft polymer chains is at least one kind selected from (meta)acrylic acid or its alkali metal salt and amine salt; itaconic acid or its alkali metal salt and amine salt; amide-based monomers; positively charged monomers having at least one kind selected from the group consisting of an ammonium group and phosphonium group; and monomers having an acid group, which is either negatively charged or to be negatively charged by dissocation, and which has a sulfonic acid group, carboxyl group, phosphoric acid group, and phosphonic acid group.

10. A surface functional member of claim 1, wherein the atom transfer radical polymerization is performed by using an organic halide or a halogenated sulfonyl compound as the initiator, and a transitional metal complex as the catalyst.

11. A surface functional member of claim 1, wherein the atom transfer radical polymerization is performed by using a polymerization initiator for free radical polymerization, and a transitional metal complex as a catalyst.

12. A surface functional member of claim 1, wherein the atom transfer radical polymerization is performed in the presence of a copper compound and an amine-based ligand as a catalyst.

13. A surface functional member of claim 1, wherein the substrate has been roughened.

14. A surface functional member of claim 1, wherein the diameter of the particles that are bondable with the ionic polar groups is in the range of 0.1 nm to 1 μm.

15. A surface functional member of claim 1, wherein the particles that are bondable with the ionic polar groups is antireflection member particles which are composed of at least one kind of pigment particles selected from the group consisting of metal oxide particles, a transparent pigment and a white pigment, and cross-linking resin particles.

16. A surface functional member of claim 1, wherein the particles that are bondable with the ionic polar groups are at least one kind selected from the group consisting of conductive resin particles, conductive or semiconductive metal particles, metal oxide particles, and metal compound particles.

17. A surface functional member of claim 1, wherein the particles that are bondable with the ionic polar groups are at least one kind of sterilizing particles selected from the group consisting of silver (Ag), copper (Cu), alloys containing at least one of silver and copper, and oxides of these metals; metal compound semiconductors, and metal compounds mixed with at least one of platinum, gold, palladium, silver, copper, nickel, cobalt, rhodium, niobium, and tin.

18. A surface functional member of claim 1, wherein the particles that are bondable with the ionic polar groups are charged particles or particles treated to have an opposite charge to the charge of the ionic polar groups to be adsorbed to the particles.

19. A surface functional member of claim 1, wherein the surface functional member is a kind selected from an antireflection member, a conductive member, a light shielding member, a surface antimicrobial member, a ultraviolet adsorbing member, a gas barrier member, an optical material, and particles for an organic electroluminescent element.
